(19) United States
(12) Patent Application Publication
(10) Pub. No.: US 2022/0090219 A1
(43) Pub. Date: Mar. 24, 2022

(54) COMPOSITIONS AND METHODS FOR EXTRACTING BIOLOGICAL MATERIAL FROM SUGAR-BEARING PLANTS

(71) Applicant: SOLENIS TECHNOLOGIES, L.P., Wilmington, DE (US)

(72) Inventor: Jonathan Bergamaschi, Wilmington, DE (US)

(73) Assignee: SOLENIS TECHNOLOGIES, L.P., Wilmington, DE (US)

(21) Appl. No.: 17/447,970

(22) Filed: Sep. 17, 2021

Related U.S. Application Data
(60) Provisional application No. 63/081,958, filed on Sep. 23, 2020.

(51) Int. Cl. C13B 10/14; C13B 50/00; C12P 7/08

(52) U.S. Cl. CPC C13B 10/14; C12P 7/08; C13B 50/00

(57) ABSTRACT

An improved process for extracting biological material from sugar-bearing plant material. In particular, the sugar-bearing plant material is treated with an extractant solution comprising water and a functionalized amphiphilic polymer, wherein the biological material is extracted from the sugar-bearing plant material. In addition, a method for producing ethanol from a sugar-bearing plant material is provided.

This application claims the benefit of U.S. Provisional application No. 63/081,958, filed 23 Sep. 2020, the entire contents of which are hereby incorporated by reference.

### TECHNICAL FIELD

An improved process for extracting biological material from sugar-bearing plant material is prov. In particular, the sugar-bearing plant material is treated with an extractant solution comprising water and a functionalized amphiphilic polymer, wherein the biological material is extracted from the sugar-bearing plant material. In addition, a method for producing ethanol from a sugar-bearing plant material is provided.

### BACKGROUND

The modern sugar and alcohol mills, known as biorefineries, are facilities that integrate physical, chemical, biochemical and thermochemical processes for converting biomass into biofuels (ethanol), energy, food (sugar, also called Pol, a term used to refer to sucrose due to its chirality, ability to deviate from the polarized light plane) and chemical/biochemical inputs (as dry yeast) in order to reduce the production of waste and emission of pollutants. An already established example of a biorefinery is the production of bioethanol and sugar with energy cogeneration from sugar cane.

Typical types of sugar cane processing units include mills, which manufacture sugar and have an attached distillery to produce ethanol from the reprocessing of molasses, and autonomous distilleries, which are dedicated exclusively to the production of ethanol. Both are energetically autonomous, generating electrical energy by burning residual biomass from production.

In general, a sugarcane biorefinery is divided into the sectors of pre-processing, sugar factory, distillery, storage, effluent treatment and utilities. In mixed biorefineries, the distillery is attached to the sugar factory and uses molasses, a by-product of sugar production, as a raw material. Pre-processing has the purpose of conditioning the cane and extracting the sugarcane juice with minimal loss of sucrose. This stage involves the reception, washing (or not), shredding and extraction of sugarcane juice. The extraction of the juice by mills, consists of mechanical axial pressure on the previously chopped and defibrated sugarcane. The defibrated cane passes through an electromagnet to remove metallic impurities and is fed to the mills through an inclined chute called Chute Donelly (or simply Donelly), whose driving forces are basically gravity and the speed at which the defibrator feeds the chute.

Two different technologies are generally used in the extraction process: grinding and diffusion. In diffusion extraction, the sucrose adsorbed to the sugarcane fibers or sugarcane bagasse (SCB), is removed by washing or leaching. In a grinding process, the juice is extracted by mechanical pressure on defibrated sugar cane leaving the SCB. The dry pulp residue that is left after the extraction of the juice from the sugar cane is known as the bagasse.

The theoretical residual of sucrose or Pol that is present in typical SCB after diffusion is about 5-6%. In the grinding or mechanical process, it can be from about 3-8%. However, despite the diffusion extraction method having a lower deviation efficiency, when compared with mechanical extraction, its implantation and maintenance costs are higher. In addition, water consumption in the diffusion process is about 3 times higher than in a mechanical process.

In most extraction plants that only work with first generation ethanol, such as sugar cane, corn, wheat, and sugar beets, after grinding/diffusion, the final SCB is sent to the utilities sector, where it is used as fuel in the boilers to produce steam and electricity to be consumed in the extraction processing operations. Therefore, the lower the percentage of residual sucrose after grinding in the last roller mill, the greater the efficiency of the plant. Many if not most biorefining mills use the milling extraction process.

Simple imbibition consists of adding water to the SCB on the feeding mat of the roller, and can be done only in the last roller (single), or in one or more of the previous rollers (double, triple, etc.). However, the imbibition method used in current biorefineries is generally compound imbibition, in which pure imbibition water is poured over the intermediate mat that feeds the last roller and the resulting broth is returned to the mat of the previous roller, and so on, until the second roller. Thus, the juice collected is the primary juice and the consolidated juice that comes out of the other rollers are recirculated to compose the compound or secondary juice. In general, the composite imbibition shows better results, in terms of extraction efficiency of total Pol (sucrose content) and water resource savings, than simple imbibition. The accumulation of primary juice and mixed juice make up the total extraction of Pol from the sugarcane juice by the mill. Table 1, provides results of actual data of accumulated juice extracted as a function of a roller, in a high efficiency biorefinery, in relation to the extraction of sugars.

TABLE 1
Accumulated total sugar extraction % in function of numbers
of roller using just water as compound imbibition.

| # of Roller | Accumulated total extraction |
|---|---|
| Roller 1 | 78.29% |
| Roller 2 | 88.35% |
| Roller 3 | 91.31% |
| Roller 4 | 93.77% |
| Roller 5 | 95.33% |
| Roller 6 | 96.98% |

*Source: Actual data from a sugarcane biorefinery in the state of São Paulo with high extraction efficiency provided to the inventor.

The proportion between the mass of water of imbibition and the mass of fiber contained in the defibrated cane (λ-lambda) must meet an optimal value, since an increase in λ improves the extraction efficiency and also increases the energy consumption necessary for the later evaporation of the juice, which increases operating costs. Studies indicate that there are no great gains in extraction efficiency when λ is greater than 3, therefore in the study below, a value of around 5 was used, for practical reasons.

Modern mills adopt practices to improve their extraction efficiency. An example is the heating of the imbibition water which can increase the total sugar extraction by about 10% when compared to cold imbibition. The heat destroys the plant tissues, exposing the cell contents and making the cane fiber more plastic and compressible, so that it is advantageous to keep the imbibition water between 70° C. and 85° C. Above that temperature, the fiber becomes slippery and causes problems with feeding (HUGOT, 1986 and PAYNE, 1990).

Another practice is the addition of imbibition water by pressurized jets on the intermediate mat that precedes each roller. As the bagasse has the capacity to absorb between 5 and 10 times its own mass in water, the addition of water or broth of imbibition in jets, prevents the superficial layer of the bagasse from absorbing all the water, allowing the passage of a portion of the water or the broth to the deeper layers, homogenizing the imbibition process, which does not occur in the addition by sprays (HUGOT, 1986).

Sugarcane biorefineries currently burn the bagasse generated after the extraction of sugars by the milling tandem system. This burning generates heat and water vapor to produce electricity. However, the extraction efficiency of bagasse sugars is physically limited to 92-97%. That means there is a potential for extraction of an additional 3-8% of total sugars that is still present in the bagasse and that is currently being burned. The present method has technical and economic importance in order to reduce sugar content into the sugarcane bagasse and consequently increase the Pol extraction in the sugarcane milling process.

Studies aimed at increasing sugar extraction in sugar plant extraction processes are based on maintaining the microbiological load of the system and/or reducing surface tension of the imbibition water. Therefore, the physicochemical principle understood in this last strategy is based on the increase of water contact angle to the natural fiber, and consequently, increase of the sucrose dissolution in water.

Accordingly, it is desirable to provide compositions and methods for improved extraction of biological materials from sugar-bearing plant material.

### SUMMARY

A composition for extracting biological material from sugar-bearing plants if provided for. The composition includes an extraction solution containing water and a functionalized amphiphilic copolymer.

Also provided, is a method relating to the extraction of biological material from sugar-bearing plant material, such as sugarcane. The extraction is carried out by contacting the sugar-bearing plant material with an extractant solution comprising water and a functionalized amphiphilic polymer wherein the functionalized amphiphilic polymer chain is a copolymer based on poly(alkylene oxide) with blocks of poly(propylene oxide)-PPO and poly(ethylene oxide)-PEO; and wherein the block copolymers distribution are according to Formula I, Formula II and Formula III:

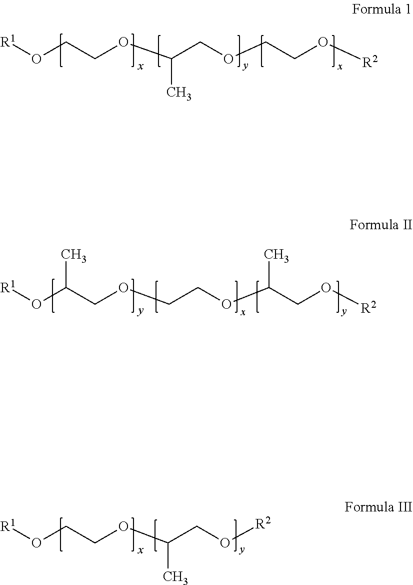

wherein ethylene oxide units of Formula I, II, and III are from about 5 wt % to about 90 wt % of the total polymer; each of x and y are independently greater than zero; and R¹ and R² are independently a radical functionalizer chosen from fatty esters and/or hydrogen. This results in increased amounts of extracted biological material from the sugar-bearing plant material.

In addition, there is provided a process for making ethanol from a sugar-bearing plant material, the process includes extracting sugar from the sugar-bearing plant material by treating the sugar-bearing plant with an extractant solution comprising water and a functionalized amphiphilic polymer and collecting the resultant juice extractant. The juice extractant is processed and fermented producing an aqueous ethanol solution. The aqueous ethanol solution is further process, isolating the ethanol from the aqueous ethanol solution.

### BRIEF DESCRIPTIONS OF THE DRAWINGS

The present method will hereinafter be described in conjunction with the following drawing figures.

FIG. 1—is a schematic view of an extraction process, i.e. sugar production process, according to one embodiment of the method;

FIG. 2—is a schematic enumeration of pilot-mill components.

FIG. 3—is a means extraction comparison chart of the new formulation with formulations currently used in the industry.

FIG. 4—is a comparation the extraction % values location and spread chart.

FIG. 5—is a outliers investigation chart.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description.

Provided is compositions and methods for increased or enhanced extraction of biological material from sugar-bearing plant material, such as sugarcane. The extraction is carried out with an extractant solution comprising water and a functionalized amphiphilic polymer. The functionalized amphiphilic polymer is a copolymer based on poly(alkylene oxide) with blocks of poly(propylene oxide)-PPO and poly(ethylene oxide)-PEO; wherein the block copolymers distribution are according to Formula I, Formula II and Formula III:

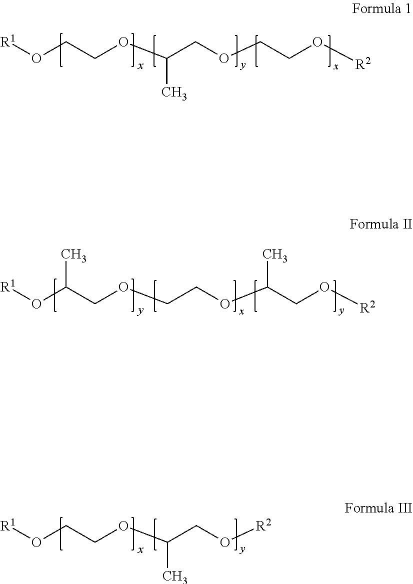

wherein ethylene oxide units of Formula I, II, and III are from about 5 wt % to 90 wt % of the total polymer; each of x and y are independently greater than zero; and R¹ and R² are independently a radical functionalizer chosen from fatty esters and/or hydrogen. The extraction solution is combined with the sugar-bearing plant material and processed. This results in increased amounts of extracted biological material from the sugar-bearing plant material compared with extractant solutions used in the industry today.

In some aspects of the current compositions and methods, the radical functionalizer is chosen from fatty esters chosen from C₄₋₃₀ monocarboxylic acid, dicarboxylic acid, tricarboxylic acid, C₁₋₃₀ monohydric alcohol, dihydric alcohol, trihydric alcohol, polyhydric alcohol compounds and combinations thereof.

In other aspects of the current compositions and methods, the fatty ester is a C₄₋₃₀ monocarboxylic acid, dicarboxylic acid, or tricarboxylic acid chosen from butyrate, caproate, laurate, myristate, monococoate, palmitate, palmitoleate, oleate, elsidiate, linoleate, linolelaidiate, arachidiate, beheniate, lignoceriate, nervoniate, stearate, isostearate, decyl oleate, sebacate, isononanoate, neopentanoate and combinations thereof.

In other aspects of the current compositions and methods, the fatty ester is a C₁₋₃₀ monohydric alcohol, dihydric alcohol, trihydric alcohol, polyhydric alcohol compounds chosen from methanol, ethanol, ethylene glycol, propylene glycol, glycerol, isopropanol, mannitol, sorbitol, silitol, maltitol, sucralose, erythritol and combinations thereof.

In yet other aspects of the current compositions and methods, the functionalized amphiphilic polymer is present in the extractant solution at a concentration of from about 0.1 parts-per-million (ppm) to about 10,000 ppm based on the weight of the extractant solution, can be from about 0.5 ppm to about 5,000 ppm, can be from about 1 ppm to about 1,000 ppm, and may be from about 5 ppm to about 100 ppm based on the weight of the extractant solution.

In other aspects of the current compositions and methods, the functionalized amphiphilic polymer has an average molecular weight of from about 1,000 g/mol to about 4,200 g/mol, can be from about 1500 g/mol to about 3500 g/mol and may be from about 2,000 g/mol to about 3,000 g/mol.

In yet other aspects of the current composition, the extractant solution further comprises sugar-bearing plant material juice, such as from sugar cane.

In other aspects of the method, there is provided a process for making ethanol from the sugar-bearing plant material. The process includes treating the sugar-bearing plant material with an extractant solution, extracting sugar from the sugar-bearing plant material forming a juice extractant. The juice extractant is further processed and fermented producing an aqueous ethanol solution. The aqueous ethanol solution is further process, isolating the ethanol from the aqueous ethanol solution.

### EXAMPLES

The following studies were done on a pilot-mill using a scaled down roller-mill that was developed by Solenis Especialidades Quimicas, Paulinia SP. The pilot-mill has a defrosted sugarcane grinding operation. In the sugar and alcohol industry, the milling is done in the juice extraction stage, which is then used to make sugar and ethanol. The operation was preceded by a sugarcane chopper and shredder, in order to break the plant cells and expose the juice contained in them, facilitating the extraction of the juice. The defibrated cane was also homogenized to condition it to feed to the pilot-mill.

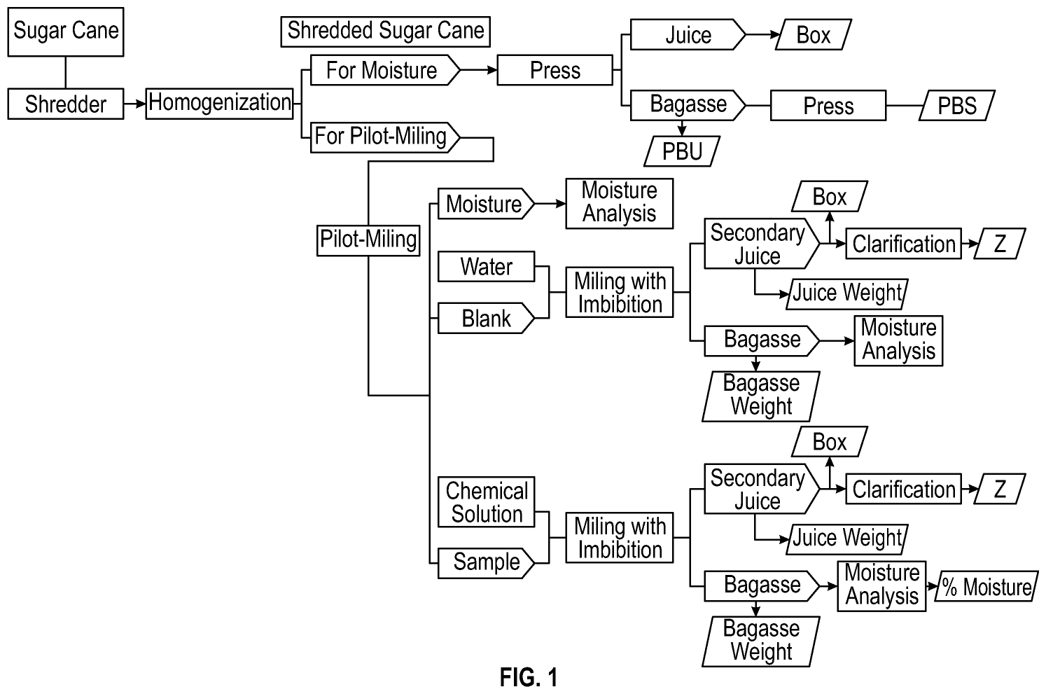

The general extracting process, as shown in FIG. 1 was used in the following examples and indicates where in the process some of the variables used in the calculations come from. The sugar-bearing plant material goes through a shredder and homogenizer wherein extractant solution, called “chemical solution” at schematic, is added to the sugar-bearing plant material. At this stage, juice extraction occurs and collected and the extractant juice further processed while the sugar-bearing plant material continues through a press section producing additional juice (brix or soluble sugar content) also called “secondary juice” and bagasse. The juice is clarified to measure ° Z (magnitude obtained in polarimeter used in calculating of sucrose amount). The bagasse was separated into a wet bagasse (PBU) and dry bagasse (PBS), wherein the dry bagasse was produced by additional drying in an oven.

### Experimental Procedure: Pilot-Milling

The experimental procedure consisted of a series of steps, each with its materials and equipment. A series of extractions were done in triplicate comparing the present formulation with known formulations and a blank or base sample for determination of the sugarcane moisture content.

It is known that water surface tension change assists in the extraction of sugar in the compression or milling stage of extraction process. Therefore, this principle was also evaluated (Blank test) in the following study. The average sugar cane composition is about 3% (W/W) proteins, starch, dextran, gums and organic acids. This could be from the functionalization of amphiphilic polymers, which potentiates the interaction of these molecules present in sugarcane with water, consequently aiding in the extraction of sugar by increasing dissolution/leaching by imbibition water.

The following data collected in this procedure will be based on the following:
Initial mass of sample (all samples)
Damp cake mass (all samples)
Dry cake batter (base sample)
° Brix of primary juice (base sample)
° Z of primary juice (base sample)
° Brix of post-digestion juice (blank and with additive samples)
° Z after digestion juice (blank and with additive samples)

### Extraction Efficiency

The sucrose extraction efficiency (E) was calculated according to the formula below and the relation between the extracted sucrose and the original sucrose content of the sugar cane (see FERNANDES, A. C. Cálculos na agroindústria de Cana-de-açúcar. 3^{a} ed. Piracicaba. STAB—Sociedade dos Técnicos Açucareiros e Alcooleiros do Brasil, 2011) using the following equation:

$$\% E = ( 1 - \frac{f}{PC} . \frac{PB}{{f}^{′}} ) \cdot 100$$

where f and f′ are the fiber percentage of the cane and the bagasse after milling, respectively. PB is the bagasse pol or bagasse sucrose amount (after milling) and PC is the cane pol or cane sucrose amount (before milling). PBU and PBS are the wet bagasse weight and the bagasse weight after drying in an oven, respectively. Variable f′ can be determined by the Tanimoto press method (see Brazilian norm ABNT 16225:2013, Determinação do teor de fibra % cana pelo método de Tanimoto), 2013, see equation below.

$$f = \frac{100 \cdot PBS - PBU \cdot Brix}{5 ( 100 - Brix )}$$

Variable f′ is calculated based on simple mass balance (see CALDAS, C. Novo manual para laboratório sucroalcooleiros. Piracicaba. STAB—Sociedade dos Tecnicos Açucareiros e Alcooleiros do Brasil, 2012), see equation below

$${f}^{′} = \frac{100 - Moisture \% bagasse - Brix}{1 - 0.01 \cdot Brix}$$

PB and PC are estimated according to the juice Pol (sucrose content) and fiber content of the bagasse and the cane, respectively, see equation below. PB is calculated by mass balance (see CALDAS, C. Novo manual para laboratório sucroalcooleiros. Piracicaba. STAB—Sociedade dos Técnicos Açucareiros e Alcooleiros do Brasil, 2012).

PB=cane juice pol(1−0,01. f′)

The determination of PC requires that a coefficient “C” is known, due to the sucrose tending to be retained on the juice rather than in the cane (see CONSECANA SP (Conselho dos produtores de cana-de-açúcar, açúcar e álcool do Estado de São Paulo). Manual de instruções. 5^{a} ed, Piracicaba, 2006; HUGOT, E. Manual da engenharia acucareira vol. 1. Sao Paulo: Ed. Mestre Jou, 1977. Traduction of I. Miocque).

Follow C coefficient and PC equation, respectively:

C=1.013−0.00575×f and PC=JC×(1-0.01×f)×C

In order to eliminate the influence of the fiber content in the extraction calculation, the Noel-Deer reduced extraction formula was used. This formula considers that a standard sugar cane has a 12.5% of fiber called the Reduced Mill Extraction (RME_{Noel Deer}) (see CALDAS, C. Novo manual para laboratorio sucroalcooleiros. Piracicaba. STAB—Sociedade dos Técnicos Açucareiros e Alcooleiros do Brasil, 2012).

$$\% ERD = 100 - \frac{( 100 - E ) \cdot ( 100 - f )}{7 \cdot f}$$

To begin the process, a known amount or weight of sugarcane was shredded and defibrillated in a Sueg model SG-D500 cane disintegrator. Homogenization can be accomplished with either a manual or electric homogenizer. 500 gram (g) samples were collected of the defibrillated sugar cane with no additives, used for moisture content calculations.

The sugar cane was pressed using a Sueg AT hydraulic press. This step was based on Brazilian norm ABNT NBR 16221: 2013. The sugar cane samples were pressed at 24.5 MPa of pressure for 1 minute. This resulted in about 70% of the juice present in the sugar cane being extracted and collected in triplicate samples. The hydraulic press removed the primary juice, measuring the Brix (use of Schmidt-Hausen refractometer) and Pol (use of ACATEC model PDA8200 polarimeter) according to Brazilian norm ABNT NBR 16223: 2013 and ABNT NBR 16224: 2013, respectively. To calculate the moisture content of the wet cake of the base sample and consequent calculation of the soaking water in relation to percent (%) fiber, the two related standards were used, ABNT NBR 16225: 2013-Tonimoto method; or NREL/TP-510-42621-thermogravimetric balance method.

### Sucrose Calculation

The apparent content of sucrose (pol) of the cane juice was determined using Octapol® clarifier according to the following equation:

juice pol=(0.99879.(° Z)+0.47374).(0,2605−0,0009882. (° Brix))

where ° Z is a measure of the polarized light deviation as determined with a polarimeter, and ° Brix is the soluble solids content measured by refractometry.

### Pilot-Milling Components

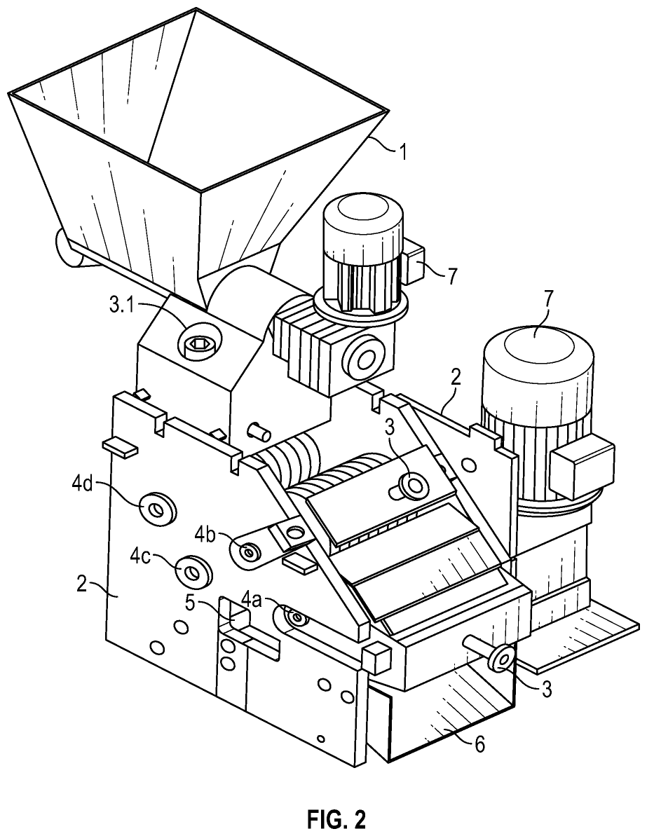

FIG. 2, is a schematic of the pilot-mill that was used in the current study. The main components of the pilot-mill used in the study include (1) feeder; (2) castle or side walls of the mill; (3) roller regulators; (3.1) water imbibition system; (4a) exit roller; (4b) upper roller; (4d) inlet roller; (4c) pressure roller and (5) axial compression base.

The pilot-mill also has two motors (7), one motor for moving the feed screw and one motor for the mill rollers. Each motor has a speed control system. The frequency of rotation of the feed thread ranged from 0 to 26 Hz and that of the milling rolls from 0 to 40 Hz.

The pilot-mill has three settings for controlling the upper roller (4b), the exit roller (4a) and axial compression base (5). Each adjustment was made through its respective regulator. The axial compression base (5) consists of chocks or stainless steel plates. The plates were fitted between the rollers and the width between the tailgate teeth and the exit roller (4a) was adjusted so that there was a sufficiently small space to allow the bagasse to pass through and compress it at the same time.

Feeding the sugar cane into the extractor was done manually. Care was taken to keep the feeder (1) constantly filled. During operation, extracted juice and bagasse would fall into a tray. The juice was drained from the back of the pilot-mill while the bagasse was collected as it came out the front (6). It was used the water imbibition system (3.1) to mimic the simple water imbibition with just water or water with the chemical additive as extractant solution. To simulate a sequence of rollers, the same sample can be reprocessed several times at the same or different settings. An external water heating unit was used to heat the imbibition water that was introduced into the pilot mill through a water imbibition water system (3.1).

### RESULTS AND DISCUSSION

The formulations used in the current study are shown in Table 2.

TABLE 2
Chemical formula of all samples used as
additives to increase sugar extraction.

| | Chemical formula of additives to |
|---|---|
| Sample | increase sugar extraction |
| New formulation | Functionalized amphiphilic polymer |
| (sample 6) | comprises blocks of poly(propylene oxide) |
| | and poly(ethylene oxide) |
| Sample 1 | Lime, milk of lime and a calcium saccharate |
| | solution |
| Sample 2 | Surfactant comprises poly(alkylene oxide) |
| | copolymer |
| Sample 3 | Hydrogen peroxide and nonionic surfactant |
| | based on ethylene oxide adduct |
| Sample 4 | Product of the condensation of an alkylene, |
| | oxide with an alkylphenol or with an |
| | aliphatic alcohol. |
| Sample 5 | Mixture of ester of sodium sulfo-succinic |
| | acid and alkylarene sulphonate |

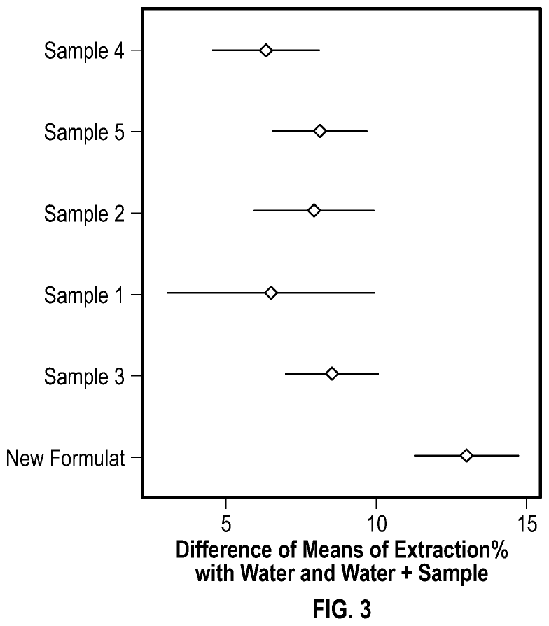

The following results are based on a difference of extraction between just water and water plus functionalized amphiphilic polymer and shows a significant improvement in sugar extraction. As can be seen from Table 3, the new formulation (Sample 6) gave the best extraction variation when compared with the formulations currently being used in extraction processes (samples 1, 2, 3, 4 and 5), i.e. higher RME_{Noel Deer} %. The FIG. 3, shows One-Way ANOVA for means of difference of extraction with water and water+sample. It indicates that there are differences among the means at the 0.05 level of significance between the formulations used in the present method and the formulations that are currently in use (P-value equals 0.002). Since the P-value is <0.05, the differences among samples are significant. The new formulation intervals do not overlap other sample intervals. Therefore, it can be concluded that there is statistically significant higher biological material extraction from the sugar-bearing plant material.

TABLE 3
Percent of Sugar Extracted from Sugar Cane

| | Difference of means of | | |
|---|---|---|---|
| | extraction % with water | Standard | Sample |
| Samples | and water + sample | deviations | Size |
| New Formulation | 13.01 | 1.26 | 4 |
| (sample 6) | | | |
| Sample 1 | 6.488 | 2.11 | 4 |
| Sample 2 | 7.98 | 1.76 | 5 |
| Sample 3 | 8.56 | 1.06 | 4 |
| Sample 4 | 6.36 | 1.27 | 4 |
| Sample 5 | 8.16 | 1.02 | 4 |

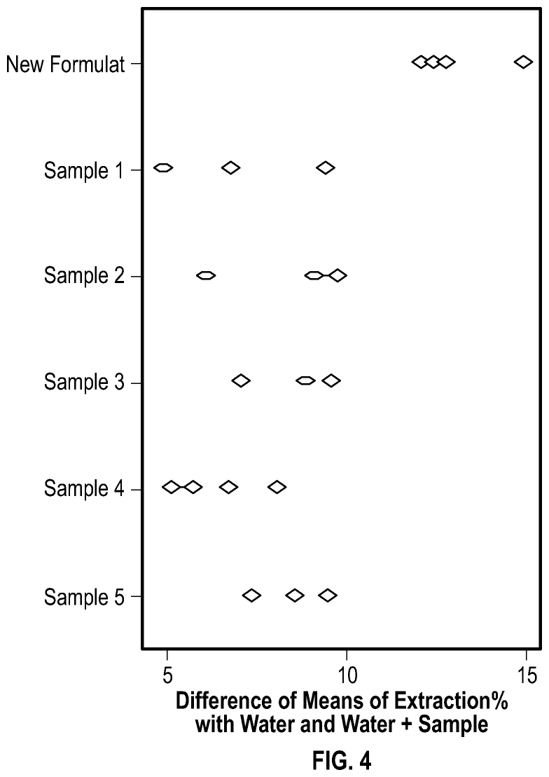

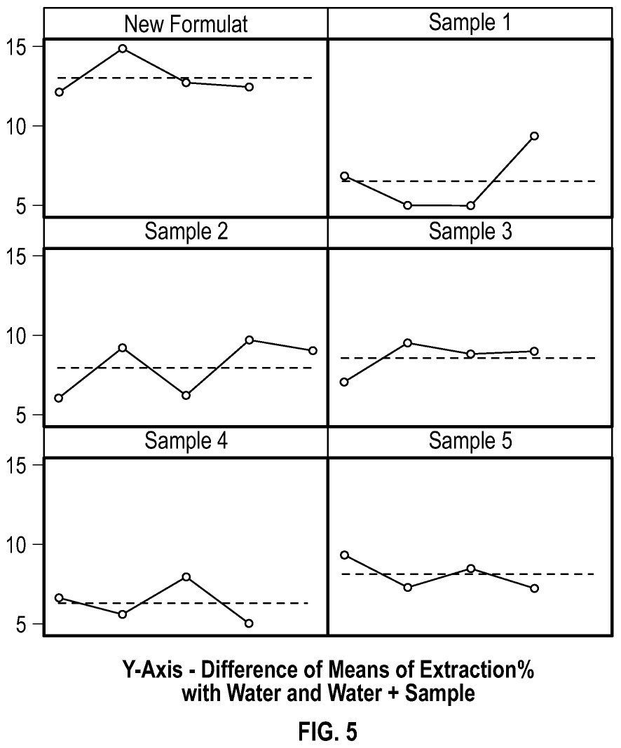

The distribution and spread of all extraction solutions used in the study were calculated and the amount of extractant solution using the present method showed a higher extraction means with a higher confidence index (see FIG. 4). As the objective of assessing the presence of any data that was inconsistent with the rest of the data set, an outlier assessment was accomplished. The evaluation indicated no outlier data was observed (see FIG. 5).

Any references cited in the present application above, including books, patents, published applications, journal articles and other publications, is incorporated herein by reference in its entirety.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment, it being understood that various changes may be made in the function and arrangement of elements described in an exemplary embodiment without departing from the scope of the invention as set forth in the appended claims and their legal equivalents.

I claim:

1. A composition for extracting biological material from sugar-bearing plants comprising: an extractant solution comprising water and a functionalized amphiphilic polymer;
wherein the functionalized amphiphilic polymer comprises blocks of poly(propylene oxide) and poly(ethylene oxide) according to Formula I, Formula II and Formula III: 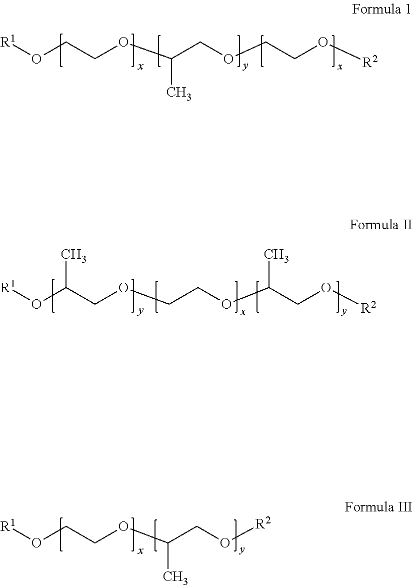
wherein ethylene oxide units of Formula I, II, and III are present in an amount of from about 5 wt % to about 90 wt % based on a total weight of the copolymer;
each of x and y is independently greater than zero; and
R¹ and R² are independently a radical functionalizer comprising fatty esters and/or hydrogen; and
sugar-bearing plants.

2. The composition of claim 1, wherein the fatty ester is chosen from C₄₋₃₀ mono-carboxylic acids, di-carboxylic acids, tri-carboxylic acids, C₁₋₃₀ mono-polyhydric alcohols, di-polyhydric alcohols, tri-polyhydric alcohol compounds, and combinations thereof.

3. The composition of claim 2, wherein the C₄₋₃₀ monocarboxylic acid, dicarboxylic acid or tricarboxylic acid is chosen from butyrate, caproate, laurate, myristate, monococoate, palmitate, palmitoleate, oleate, elsidiate, linoleate, linolelaidiate, arachidiate, beheniate, lignoceriate, nervoniate, stearate, isostearate, decyl oleate, sebacate, isononanoate, neopentanoate and combinations thereof.

4. The composition of claim 2, wherein the C₁₋₃₀ monohydric, dihydric, trihydric or polyhydric alcohol compound is chosen from methanol, ethanol, ethylene glycol, propylene glycol, glycerol, isopropanol, mannitol, sorbitol, silitol, maltitol, sucralose, erythritol and combinations thereof.

5. The composition of claim 1, wherein the functionalized amphiphilic polymer comprises about 0.1 parts-per-million (ppm) to about 10,000 ppm by weight of the extractant solution

6. The composition of claim 1, wherein the functionalized amphiphilic polymer has an average molecular weight of at least about 1,000 g/mol to about 4,200 g/mol.

7. A process for extracting biological material from sugar-bearing plant material comprising the steps of:
contacting the sugar-bearing plant material with an extractant solution comprising water and a functionalized amphiphilic copolymer; and
extracting the biological material from the sugar-bearing plant material;
wherein the functionalized amphiphilic copolymer comprises blocks of poly(propylene oxide) and poly(ethylene oxide) according to Formula I, Formula II and Formula III: 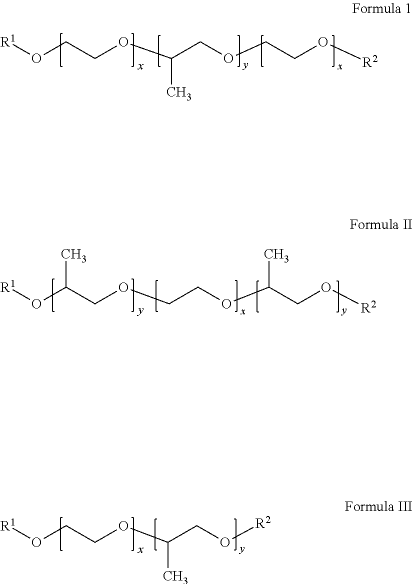
wherein ethylene oxide units of Formula I, II, and III are present in an amount from about 5 wt % to about 90 wt % based on a total weight of the polymer;
each of x and y is independently greater than zero; and
R¹ and R² are independently a radical functionalizer comprising fatty esters and/or hydrogen.

8. The process of claim 7, wherein the fatty ester is chosen from C₄₋₃₀ mono-carboxylic acids, di-carboxylic acids, tri-carboxylic acids, C₁₋₃₀ mono-polyhydric alcohols, di-polyhydric alcohols, tri-polyhydric alcohol compounds, and combinations thereof.

9. The process of claim 7, wherein the C₄₋₃₀ monocarboxylic acid, dicarboxylic acid or tricarboxylic acid is chosen from butyrate, caproate, laurate, myristate, monococoate, palmitate, palmitoleate, oleate, elsidiate, linoleate, linolelaidiate, arachidiate, beheniate, lignoceriate, nervoniate, stearate, isostearate, decyl oleate, sebacate, isononanoate, neopentanoate and combinations thereof.

10. The process of claim 7, wherein the C₁₋₃₀ monohydric, dihydric, trihydric or polyhydric alcohol compound is chosen from methanol, ethanol, ethylene glycol, propylene glycol, glycerol, isopropanol, mannitol, sorbitol, silitol, maltitols, sucralose, erythritol and combinations thereof.

11. The process of claim 7, wherein the functionalized amphiphilic polymer comprises about 0.1 parts-per-million (ppm) to about 10,000 ppm by weight of the extractant solution.

12. The process of claim 7, wherein the functionalized amphiphilic polymer comprises about 5 ppm to about 100 ppm by weight of the extractant solution.

13. The process of claim 7, wherein the functionalized amphiphilic polymer has an average molecular weight of at least about 1,000 g/mol to about 4,200 g/mol.

14. A process for producing ethanol from a sugar-bearing plant material, said method comprising:
contacting the sugar-bearing plant material with an extractant solution comprising water and a functionalized amphiphilic polymer;
extracting sugar from the sugar-bearing plant material;
processing and fermenting the sugar-bearing extractant solution to form an aqueous solution comprising ethanol; and
isolating the ethanol from the aqueous solution,
wherein the functionalized amphiphilic polymer chain is a copolymer based on poly(alkylene oxide) with blocks of poly(propylene oxide)-PPO and poly(ethylene oxide)-PEO; wherein the blocks copolymer distribution are according to Formula I, Formula II and Formula III: 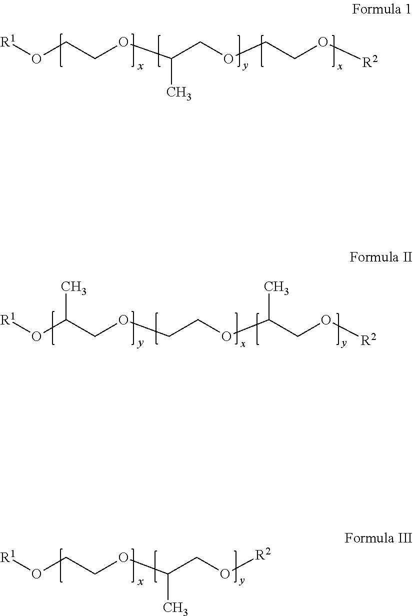
wherein the ethylene oxide units of Formula I, II, and III are from about 5 wt % to 90 wt %;
each of x and y are independently greater than zero; and
R¹ and R² are independently a radical functionalizer comprising fatty esters and/or hydrogen.

15. The process of claim 14, wherein the fatty ester is chosen from C₄₋₃₀ mono-carboxylic acids, di-carboxylic acids, tri-carboxylic acids, C₁₋₃₀ mono-polyhydric alcohols, di-polyhydric alcohols, tri-polyhydric alcohol compounds, and combinations thereof.

16. The process of claim 15, wherein the C₄₋₃₀ monocarboxylic acid, dicarboxylic acid or tricarboxylic acid is chosen from butyrate, caproate, laurate, myristate, monococoate, palmitate, palmitoleate, oleate, elsidiate, linoleate, linolelaidiate, arachidiate, beheniate, lignoceriate, nervoniate, stearate, isostearate, decyl oleate, sebacate, isononanoate, neopentanoate and mixtures thereof.

17. The process of claim 15, wherein the C₁₋₃₀ monohydric, dihydric, trihydric or polyhydric alcohol compound is chosen from methanol, ethanol, ethylene glycol, propylene glycol, glycerol, isopropanol, mannitol, sorbitol, silitol, maltitols, sucralose, erythritol and mixtures thereof.

18. The process of claim 14, wherein the functionalized amphiphilic polymer comprises about 0.1 parts-per-million (ppm) to about 10,000 ppm by weight of the extractant solution.

19. The process of claim 14, wherein the functionalized amphiphilic polymer comprises about 5 ppm to about 100 ppm by weight of the extractant solution.

20. The process of claim 14, wherein the functionalized amphiphilic polymer has an average molecular weight of at least about 1,000 g/mol to about 4,200 g/mol.
